(19) United States
(12) Patent Application Publication
(10) Pub. No.: US 2024/0050070 A1
(43) Pub. Date: Feb. 15, 2024

(54) ULTRASONIC MECHANICAL 3D IMAGING PROBE WITH SELECTABLE ELEVATION FOCUS

(71) Applicant: KONINKLIJKE PHILIPS N.V., EINDHOVEN (NL)

(72) Inventors: Andrew Robinson, Kenmore, WA (US); Changhong Hu, Bothell, WA (US)

(21) Appl. No.: 18/267,129

(22) PCT Filed: Dec. 8, 2021
(86) PCT No.: PCT/EP2021/084778

§ 371 Date: Jun. 14, 2023

Related U.S. Application Data
(60) Provisional application No. 63/125,420, filed on Dec. 15, 2020.

(51) Int. Cl. A61B 8/08; G01S 7/52; G01S 15/89; A61B 8/00

(52) U.S. Cl. CPC A61B 8/483; G01S 7/52057; G01S 15/8993; G01S 15/8945; A61B 8/466; A61B 8/4494; A61B 8/4488

(57) ABSTRACT

An ultrasonic diagnostic imaging system produces 3D images by scanning a target volume with a mechanical probe, which scans the target volume by sweeping the scan plane of an array transducer through the target volume in an elevation direction. The array transducer has two selectable focal depths, and a plurality of scan planes of image data are acquired with a far field focus, and a plurality of scan planes of image data are acquired with a near field focus. The scan planes acquired with the far field focus are separated in the elevation direction by a distance which satisfies a spatial sampling criterion in the far field, and the scan planes acquired with the near field focus are separated in the elevation direction by a distance which satisfies the spatial sampling criterion in the near field, resulting in fewer scan plane acquisitions with the near field focus that the number of scan plane acquisitions with far field focus and hence an improved volume rate of display.

This invention relates to 3D medical ultrasound probes and, in particular, to 3D imaging probes in which a transducer array is mechanically swept across a 3D image field, and the transducer array has a selectable elevation focus.

Real time ultrasound imaging became possible many years ago with the development of several types of imaging probes. Mechanical sector-scanning probes used a motorized mechanism to oscillate a transducer element back and forth to scan a sector-shaped image field. Phased array probes used phased actuation of the elements of a linear array transducer to scan a sector-shaped image plane. Linear array probes actuated successive groups of transducer elements along an array to scan a rectangular image plane. Mechanical probes traded off system complexity for the reliability concern of motorized mechanisms, while phased array probes traded off motorized mechanisms for system beamformer complexity. Linear array probes were intermediate, lacking motorized mechanisms while requiring simpler beamforming and element switching.

As ultrasound system imaging performance improved, developers began to consider how these probe types could be modified to perform three dimensional (3D) imaging. Eventually two approaches to 3D imaging probes became widely accepted. One was an evolution of the phased array approach, in which a two-dimensional (2D) array of transducer elements is scanned by phased transmission and reception, enabling beam steering and focusing in three dimensions over a volumetric target region. The enabling technology for such matrix array probes was the microbeamformer, whereby control of beam transmission and reception is provided by semiconductor devices inside the probe. The other approach which took hold was an evolution of mechanical scanning, whereby an array transducer is swept back and forth to sweep its scan plane through the target volume. The image data from the image planes acquired during the mechanical sweep are then processed together to produce a 3D image of the swept volume.

However, the image plane data of the mechanically swept array of a mechanical 3D imaging probe, using a conventional beamformer for a 2D image plane, is only focused in the image plane. There is no focusing in the elevation dimension between the image planes. To provide such elevation focusing it would be necessary to add transducer elements in the elevation dimension, which adds the complexity of 2D array functionality to the beamformer; the advantage of beamformer simplicity otherwise inherent in mechanical probe implementations is lost, and the probe mechanical complexity remains. Accordingly, it is desirable to realize a mechanical 3D probe design that utilizes a simple beamformer implementation but still affords beam focusing in the elevation dimension. It is further desirable to do this while providing high volume frame rates of display.

In accordance with the principles of the present invention, a mechanical 3D imaging probe is provided which provides static elevation focusing in both the near field and the far field by scanning one set of planes with a near field elevation focus and another, interleaved, set of planes with a far field elevation focus. The scanned planes of the far field focused set are spaced apart by distances which satisfy a desired spatial sampling criterion in the far field and the scanned planes of the near field focused set are spaced apart by distances which satisfy the desired spatial sampling criterion in the near field. Preferably scan plane spacing is uniform within each set of scan planes. A constructed implementation of the invention thereby adequately spatially samples the target volume in both the near and far fields with both near and far field focusing, and provides a high volume rate of display by reducing unneeded scan plane acquisitions.

In the drawings:

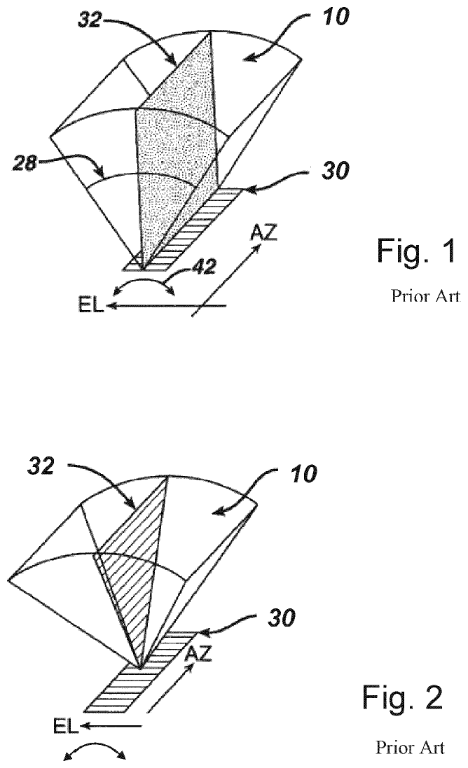

FIG. 1 illustrates the linear scanning of a rectangular image plane with a 1D (one-dimensional) array transducer which is rocked back and forth to acquire image data over a wedge-shaped scan volume.

FIG. 2 illustrates the phased scanning of a sector-shaped image plane with a 1D array transducer which is rocked back and forth to acquire image data over a pyramidal-shaped scan volume.

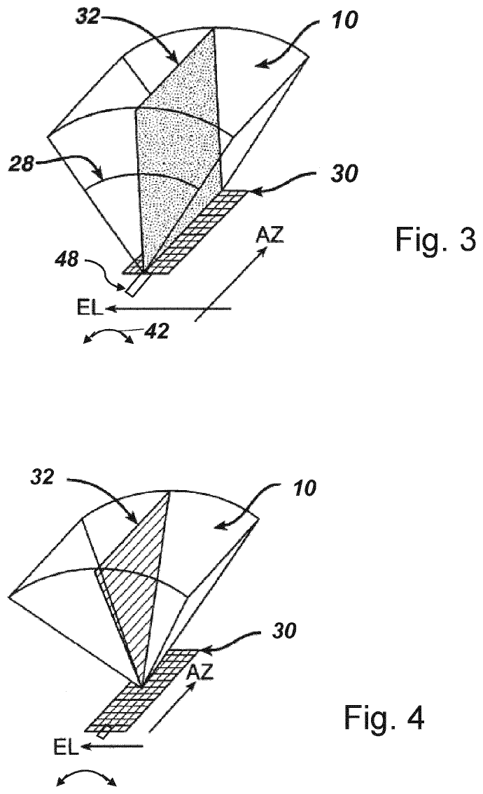

FIG. 3 illustrates the linear scanning of a rectangular image plane with a 1xD array transducer which is rocked back and forth to acquire image data over a wedge-shaped scan volume.

FIG. 4 illustrates the phased scanning of a sector-shaped image plane with a 1xD array transducer which is rocked back and forth to acquire image data over a pyramidal-shaped scan volume.

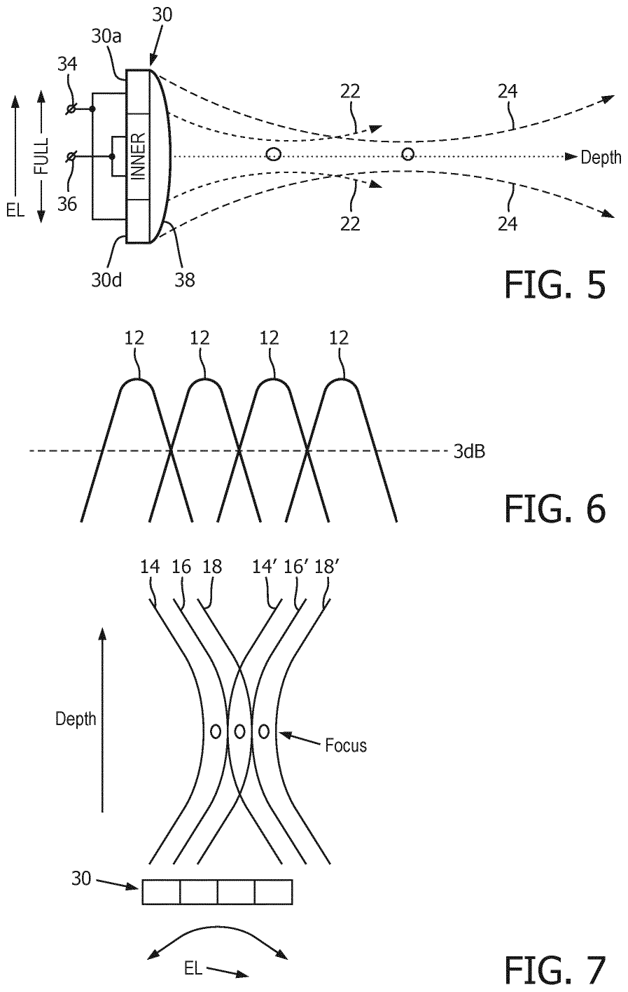

FIG. 5 illustrates the construction and operation of a 1xD array transducer to provide selectable near or far field elevation focusing in accordance with the principles of the present invention.

FIG. 6 illustrates, from an axial beam view, the spacing of scan planes of a mechanical 3D imaging probe necessary to meet a desired spatial sampling criterion in the elevation dimension in accordance with the principles of the present invention.

FIG. 7 illustrates, in a depth-of-field view, the spacing of scan planes of a mechanical 3D imaging probe necessary to meet a desired spatial sampling criterion in the elevation dimension in accordance with the principles of the present invention.

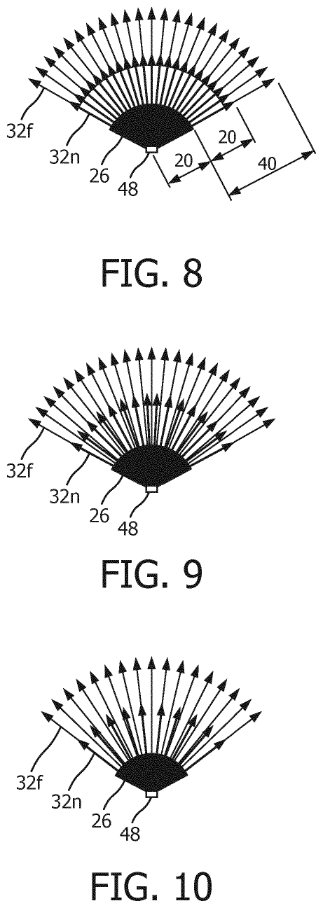

FIG. 8 illustrates the mechanical scanning of a target volume with adequate spatial sampling with a 1xD array transducer with each far field focused image plane in alignment with a near field focused image plane.

FIG. 9 illustrates the mechanical scanning of a target volume with a 1xD array transducer with independent adequate spatial sampling for the far and near field focused image planes.

FIG. 10 illustrates the mechanical scanning of a target volume with a 1xD array transducer with a tighter radius of curvature than that of FIG. 9, with independent adequate spatial sampling for the far and near field focused image planes.

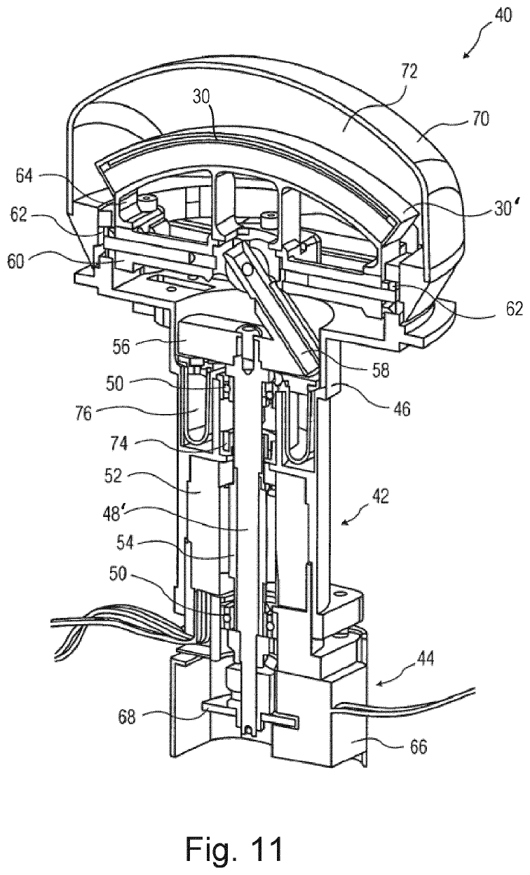

FIG. 11 illustrates the motor-driving scanning mechanism of a mechanical 3D ultrasound probe.

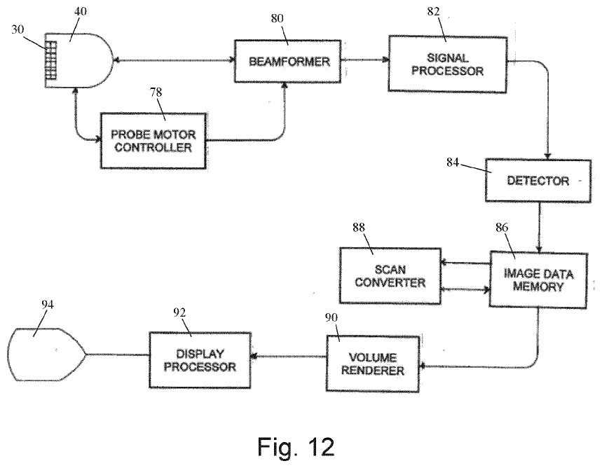

FIG. 12 illustrates in block diagram form an ultrasound system constructed in accordance with the principles of the present invention.

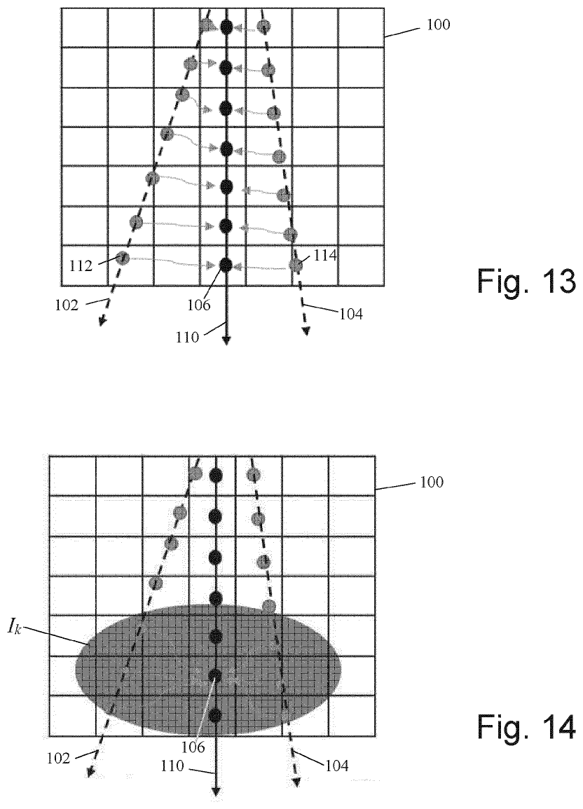

FIG. 13 illustrates one technique for performing scan conversion in the ultrasound system of FIG. 12.

FIG. 14 illustrates a second technique for performing scan conversion in the ultrasound system of FIG. 12.

Referring first to FIG. 1, the mechanical scanning of a 3D target volume 10 by rocking a linearly scanned 1D array transducer 30 is illustrated. The 1D array transducer 30 is rocked back and forth to sweep the array's two dimensional scan plane 32 in an arcuate path through the volume 10 as illustrated by arrow 42. At specified points in the arcuate path of motion, the array transducer is actuated to scan an image plane and acquire image data. The scanning of each scan plane position is performed electronically in the azimuth dimension (AZ) as the scan plane is rocked back and forth in the arcuate elevation dimension (EL). Scanlines may be continuously acquired in the azimuth direction as the array is swept in elevation, and the resultant planes may be slightly curved or angled due to the mechanical motion; as long as the spatial location of each scanline is known, a volume image may be accurately reconstructed. After scan planes of image data have been acquired across the entire wedge-shape volume 10, the image data of all of the acquired image planes is processed to produce a 3D image of the volume 10.

FIG. 2 illustrates substantially the same scanning technique as the example of FIG. 1, except that in this case each scan plane 32 is not linearly scanned, but is scanned by phased steering of beams over a sector-shaped scan plane 32. All of the transmit and receive beams of the scan plane emanate from the same point on the surface of the array 30, the apex of the inverted pyramidal volume 10. As the transducer array 30 is rocked back and forth as indicated by the arrow at the bottom of the drawing, successive scan planes 32 of image data are acquired over the arcuate span of the volume 10 in the elevation dimension. The image data of all of the acquired image planes is processed to produce a 3D image of the pyramidal volume 10.

FIG. 3 illustrates the linear scanning of a wedge-shaped volume 10 by scan planes 32 of an array transducer 30 which in this example is a 1xD array transducer. The “D” notation is an industry-wide designation of different transducer array configurations with different operating characteristics. For instance, the “2D” designation indicates an array transducer with elements extending in two dimensions and which can be electronically steered and focused in both azimuth (AZ) and elevation (EL). A 1.75D array transducer can be steered and focused in azimuth and minimally steered and focused in elevation. A 1.5D array transducer can be steered and focused in azimuth, and has dynamic elevation focusing with no elevation steering. Designations below 2D are typically characterized by fewer elements in the elevation dimension than the azimuth dimension, or the inability to operate transducer elements independently, e.g., elements are electrically connected together and operated in unison. A 1D array has multiple elements in only the azimuth dimension and has no electronic steering or focusing in the elevation dimension. The “x” in the 1xD designation indicates that the array has very few elements in the elevation dimension, typically 25% or less of the number of elements in the azimuth dimension. This provides a small but noticeable ability to provide elevation focusing. The scanning configuration of FIG. 3 is like that of FIG. 1, except that the acquired image planes exhibit some focusing in the elevation dimension due to the use of a 1xD transducer array 30. FIG. 4 operates in the same manner as the FIG. 2 configuration, except that each scan plane can be minimally focused in elevation by the 1xD transducer array.

FIG. 5 illustrates a 1xD transducer array 30 configured for operation in accordance with the principles of the present invention. This cross-sectional view illustrates the extent of the array in the elevation dimension, which in this example is four transducer elements wide, with end elements 30a and 30d and two inner elements between them. The emitting face of the array is covered with a lens 38 which provides a small degree of fixed elevation focus. The number of such four-element rows in the azimuth dimension is a larger number such as 128 or 196, for instance. The two outer elements are electrically connected together and to a terminal 34 so that the two outer elements 30a and 30b are operated in unison. The two inner elements are also electrically connected together and to a terminal 36. When just the inner element terminal 36 is used in operation, only the inner elevation aperture of two elements is active. The inner aperture exhibits a transmit and receive beam profile as indicated by the dotted lines 22. The circle at the narrowest approach of the beam profile lines 22 to each other marks the point of maximum focus of the inner aperture. When both terminals 34 and 36 are operated together, as by coupling them together or pulsing terminal 34 just prior to terminal 36 and receiving with all four elements, the full elevation aperture of four elements is active. The full aperture in this example exhibits a beam profile as indicated by dashed lines 24, again with a maximum focus marked by the circle at the narrowest approach of profile lines 24. Successive use of the inner and full apertures will thus acquire two scanlines of image data, one with a near field focus and another with a far field focus. The curve 28 in FIG. 3 marks the half-depth of each plane 32 that scans the volume 10, and the beam profiles of FIG. 5 would locate one focal point in the shallow half of that depth, and another focal point in the deeper half. The two scanlines of FIG. 5 can be combined, as by compounding the data or using shallow and deep zones to produce a scanline of image data with both near and far field focal properties. For 3D imaging, this can be done by scanning with the full array to acquire image data with a far field focus, and then with the inner aperture to acquire scan plane data with a near field focus, producing two scan planes of image data with both near and far field focal properties, an improvement over acquisition of an image with only a single focal depth. The dotted line extending from the centers of the two apertures in the Depth dimension marks the center of beams transmitted and received by the transducer array and hence the center of the image plane when the full transducer array is used to scan a scan plane in the azimuth dimension.

When successive scanlines are acquired adjacent to each other in azimuth in order to scan an image plane, or image planes are acquired adjacent to each other in elevation in order to scan a 3D volume, it is important that the scanlines or planes be close enough together so that the image field is not spatially undersampled. If the gaps between scanlines or planes are large, there will be no or negligible echo signal energy returning from the undersampled regions and undersampling artifacts can appear in the image. Typically these artifacts manifest themselves as “jailbar” artifacts, faint lines which run through the resultant image. The appearance of these artifacts and whether they are objectionable or not is somewhat subjective and due to numerous factors. Other system image processing and filtering can reduce artifact levels, and artifacts which may be objectionable to one viewer may be unobjectionable to another. A typical approach to minimize artifacts is to set the beam or plane spacing, as indicated by beam profile adjacency or overlap, which reduces spatial sampling artifacts below an objectionable level. This, of course, is a matter of design choice. FIG. 6 gives one example of beam or plane spacing, in which the ultrasound system designer has chosen beams or planes to be immediately adjacent at a −3 dB level of acoustic energy roll off. Other designers may choose a −2 dB level or a −4 dB level, for instance. In the example of FIG. 6 the beams or image planes are normal to the drawing sheet with the peaks of the energy profiles marking the locations of the beam or plane centers. As the dashed line marking the intersection of the skirts of the profiles shows, the beams or planes are immediately adjacent to each other at the −3 dB level.

FIG. 7 illustrates a longitudinal view of beam or plane energy profile spacing in the depth dimension. In this example the array transducer 30 has scanned three adjacent beams or planes with respective energy profiles 14-14′, 16-16′, and 18-18′ from three different array positions in the case of a mechanically scanned 1xD array transducer 30. The circles at the intermediate depth mark the maximum focal points of the three profiles. As seen in the drawing, the energy profiles are just touching each other at the focal depth. If the energy profiles are −3 dB profiles, then the beams or planes are immediately adjacent to each other at the −3 dB level at the depth of maximum focus to provide the desired degree of spatial sampling and artifact reduction.

As discussed in conjunction with FIG. 5, a 1xD array can be operated to provide image data over a full depth of field with two focal regions, one in the near field and one in the far field. While a better focused image will result, the drawback is that two transmit-receive cycles must be used for each beam or scan plane, one with the transducer array operating with the inner aperture for near field focusing and another with the transducer array operating with the full aperture for far field focusing. For 3D volume scanning, this means that the time required to acquire the image data for a full volume image has doubled compared to a single focus, which halves the volume rate of display. It would be desirable, of course, to produce the better focused image but without a halving of the display rate. In accordance with the principles of the present invention, this may be accomplished as illustrated in FIGS. 8, 9, and 10. In these examples a curved 1xD transducer array, curved in the azimuth (long) dimension, is mechanically scanned from left to right. These drawings illustrate the sweep of the array in the elevation direction; the azimuth dimension of the array, in which the array is curved, is normal to the plane of the drawing. The transducer array travels in the arcuate path at the top of the dark sector 26 at the bottom of each drawing which may, in a constructed implementation, be an array mount assembly which carries the transducer array through its arc of travel. A curved array transducer is used because it can scan a wider field of view provided by its curvature, which widens the field of view mechanically without the need to burden the beamformer with excessive beam steering requirements. The curved array transducer is configured to be set to focus at one of two different focal depths as illustrated in FIG. 5.

The conventional way to scan a volume field with such an arrangement is illustrated in FIG. 8. In this drawing, each arrow represents the location of an image plane center along the arc of travel, which in this example is 130°. The radius of curvature from the virtual apex 48 to the arc of travel at the top of the dark sector 26 is 20 mm in this example. The locations along the 130° arc of travel where image planes are acquired by the array transducer are marked by arrows, the darker arrows 32n marking near field focus image planes and the lighter arrows 32f marking far field focus image planes. In this example the focal depth of the near field focused planes 32n is at a depth of 20 mm, and the focal depth of the far field focused planes 32f is 40 mm. Due to the arcuate path of transducer array travel, acquired image planes will be more widely separated in the far field than in the near field. This makes the far field the more critical for determination of adequate spatial sampling. In this example the system designer has calculated the spacing between far field focused planes that provides a desired degree of spatial sampling artifact reduction, as discussed above, and determined that 25 evenly spaced image planes will adequately spatially sample the far field of the image region, a spacing of 5.2° between image plane acquisitions. This sets the positions in the target volume where 25 near field focused image planes are acquired, with the near field focused image planes 32n aligned with those of the far field, as shown by the alignment of the near and far field arrows in FIG. 8.

However, if the inter-plane spacing in FIG. 8 is sufficient in the far field to provide the desired reduction of spatial sampling artifacts, the near field planes in the same angular locations are spatially oversampling the near field. To improve the volume rate of display, the designer should perform a second calculation for adequate spatial sampling in the near field. When the same spatial sampling criterion (e.g., −3 dB) is used for the near field calculation as was used for the far field, the number of image planes required to adequately sample the near field is 16 in this example. The near field image plane acquisitions 32n should be evenly distributed along the 130° arc of travel, as illustrated in FIG. 9, in this example being separated by an arc of travel of 8⅛°. It is seen that this necessarily means that the near field and far field scan planes are no longer spatially aligned, a situation which can be properly handled by display point interpolation during scan conversion as discussed below. In this example, it is seen that separate determinations of inter-plane spacings has resulted in a reduction of the number of scan plane acquisitions for a volume image from 50 planes to 41 planes. This is a reduction in the scan plane acquisition time of 18%, a savings which translates directly into an improvement in the volume rate of display, important for real time volume imaging.

The present inventors have determined that the degree of volume frame rate improvement for an implementation of the present invention is related to the degree of curvature of the curved array transducer: the more tightly curved the radius of curvature of the array, the greater the degree of improvement in display rate. For a planar (flat) array such as that shown in FIGS. 1 and 2, the radius of curvature is infinite, and there is no improvement from separate spatial sampling calculations. A lesser radius of curvature will result in greater display rate improvement as illustrated by the example of FIG. 10. In the examples of FIGS. 8 and 9, the curved array exhibits a radius of curvature of 20 mm, depicted by the distance from the virtual apex 48 to the top of the dark sector 26. In the example of FIG. 10, the transducer array exhibits a radius of curvature of 10 mm. Applying the same spatial sampling criterion in the spatial sampling calculation as was used for FIGS. 8 and 9 and with the same near and far field focal depths, the calculations for inter-plane spacing in FIG. 10 yield a determination of seventeen scan planes needed to adequately spatially sample the far field and ten scan planes for the near field. As compared with using the same number of scan planes for each focal depth, there is a savings of seven scan plane acquisitions, an improvement in image data acquisition time of over 20%.

FIG. 11 is a cross sectional isometric view of the scanning mechanism of a 3D mechanical probe 40 suitable for mechanically oscillating a curved array transducer in a scanning arc for 3D imaging. The probe 40 includes a positional actuator 42 that is mechanically coupled to the transducer assembly 30 and a positional sensor 44. The transducer assembly 30′, the positional actuator 42 and the positional sensor 44 are positioned within a supporting structure 46. The positional actuator 42 includes a drive shaft 48′ that extends upwardly from the positional sensor 44 along a longitudinal axis of the probe 40. The drive shaft 48′ is rotationally supported within the supporting structure 46 of the probe 40 by bearings 50 positioned near respective ends of the drive shaft 48′. The positional actuator 42 also includes an armature structure 52 that is stationary with respect to the supporting structure 46, and a permanent magnet field structure 54 coupled to the drive shaft 48′. When the armature structure 52 is selectively energized, a torque is developed that rotates the drive shaft 48′ in a desired rotational direction so that the drive shaft 48′ and the field structure 54 form a driven member. The armature structure 52 may also be selectively energized to rotate the drive shaft 48 in increments of less than a full rotation, and at different rotational rates during the rotation of the drive shaft 48.

The positional actuator 42 further includes a crank member 56 that is coupled to the drive shaft 48′, which rotatably couples to a lower, cylindrical-shaped portion of a connecting member 58. The relative position of the crank member 56 with respect to the supporting structure 46 allows adjustment of the mechanical sweeping range of the transducer array assembly 30′. An upper end of the connecting member 58 is hingedly coupled to a pivot member 60 that is axially supported on the structure 46 by a pair of bearings 62. The pivot member 60 further supports a cradle 64 that retains the transducer assembly 30′. Although not shown in FIG. 11, the cradle 64 may also include electrical contacts so that individual elements of the curved array transducer 30 may transmit and receive ultrasonic signals, as previously described above. The contacts may further be coupled to a conductive assembly, such as a flex circuit, that is coupled to a beamformer 80, as shown in FIG. 12. In operation, rotational motion imparted to the crank member 56 by the drive shaft 48′ produces an oscillatory motion in the pivot member 60, which permits the transducer assembly 30′ and transducer array 30 to be moved through a selected scan angle. Further details of the scanning mechanism of FIG. 11 may be found in US Pat. Pub. No. 2004/0254466 (Boner et al.)

The positional sensor 44 includes a counter 66 that is stationary with respect to the supporting structure 46, and an encoding disk 68 that is fixedly coupled to the drive shaft 48′, so that the encoding disk 68 and the drive shaft 48′ rotate in unison. The encoding disk 68 includes a plurality of radially-positioned targets that the counter 66 may detect as the encoding disk 68 rotates through a gap in the counter 66, thus generating a positional signal for the shaft 48′. Since the angular position of the transducer array 30 may be correlated with the rotational position of the shaft 48′, the encoding disk 68 and the counter 66 therefore cooperatively form a sensor capable of indicating the angular orientation of the transducer array 30. In one particular implementation, the encoding disk 68 and the counter 66 are configured to detect the rotational position of the drive shaft 48′ by optical means. The disk 68 and the counter 66 may also be configured to detect the rotational position of the drive shaft 48′ by magnetic means, and still other means for detecting the rotational position of the drive shaft 48′ may also be used.

Still referring to FIG. 11, the probe 40 further includes a cover 70 that is coupled to the supporting structure 46. The cover 70 is formed from a material that is acoustically transparent at ultrasonic frequencies. The cover 70 further partially defines an internal volume 72 that sealably retains an acoustic coupling fluid (not shown) that permits ultrasonic signals to be exchanged between the transducer assembly 30 and the cover 70 by providing a suitable acoustic impedance match. In one aspect, a silicone-based fluid may be used that also provides lubrication to the mechanical elements positioned within the volume 72. A shaft seal 74 is positioned within the supporting structure 46 that surrounds the drive shaft 48′ to substantially retain the acoustic coupling fluid within the volume 72. The internal volume 72 further includes an expandable bladder 76 that is positioned below the crank member 56 to permit the fluid retained within the volume 72 to expand as the fluid is heated or exposed to low pressure, thus preventing leakage of the fluid from the volume 72 that may result from excessive fluid pressures developed within the probe 40.

Referring to FIG. 12, an ultrasonic diagnostic imaging system constructed in accordance with the principles of the present invention is shown in block diagram form. A 3D mechanical transducer scanning mechanism such as that shown in FIG. 11 which scans with a curved 1xD transducer array 30 is provided in an ultrasound probe 40 for transmitting ultrasonic waves and receiving echo information. The transmission of ultrasonic beams from the transducer array 30 is directed by a beamformer 80 coupled to the probe. Among the transmit characteristics controlled by the beamformer are the number, spacing, amplitude, phase, frequency, polarity, and diversity of transmit waveforms. Also coupled to the 3D mechanical probe 40 is a probe motor controller 78. The probe motor controller is coupled to the armature structure 52 of the probe to control the direction, speed, and incremental steps of motor actuation. The probe motor controller is also coupled to the beamformer for coordination of transducer array motion and scan actuation by providing positional signals from the counter 66 to the beamformer 80. The beamformer can thereby actuate the transducer array to acquire a scan plane of image data each time the transducer array is in a proper orientation for scan plane data acquisition. The echo signals received by elements of the transducer array 30 are beamformed by appropriately delaying them and then combining them.

The coherent echo signals produced by the beamformer 80 undergo signal processing by a signal processor 82, which includes filtering by a digital filter and noise or speckle reduction as by spatial or frequency compounding. The digital filter of the signal processor 82 can be a filter of the type described in U.S. Pat. No. 5,833,613 (Averkiou et al.), for example.

The beamformed and processed coherent echo signals are coupled to a detector 84. The detector may perform amplitude (envelope) detection for a B mode image of structure in the body such as tissue. The B mode processor performs amplitude detection of quadrature demodulated I and Q signal components by calculating the echo signal amplitude in the form of (I²+Q²)^{1/2}. The quadrature echo signal components may also be used for Doppler flow or motion detection. For Doppler processing, the detector 84 stores ensembles of echo signals from discrete points in an image field which are then used to estimate the Doppler shift at points in the image with a fast Fourier transform (FFT) processor. The rate at which the ensembles are acquired determines the velocity range of motion that the system can accurately measure and depict in an image. The Doppler shift is proportional to motion at points in the image field, e.g., blood flow and tissue motion. For a color Doppler image, the estimated Doppler flow values at each point in a blood vessel are wall filtered and converted to color values using a look-up table. The wall filter has an adjustable cutoff frequency above or below which motion will be rejected such as the low frequency motion of the wall of a blood vessel when imaging flowing blood. The B mode and Doppler signals are stored in an image data memory 86 in association with the spatial coordinates in the target volume from which they were acquired.

The B mode image signals and Doppler flow or motion values stored in memory are coupled to a scan converter 88 which converts the B mode and Doppler samples from the radial coordinates by which they were acquired to Cartesian (x, y, z) coordinates for display in a desired display format, e.g., a rectilinear volume display format or a sector or pyramidal display format. Either a B mode image or a Doppler image may be displayed alone, or the two shown together in anatomical registration in which the color Doppler display values show the blood flow in tissue and vessels in the image. The scan converted volume image data, now associated with x, y, z Cartesian coordinates, is coupled back to the image data memory 86, where it is stored in memory locations addressable in accordance with the spatial locations from which the image values were acquired. The image data from 3D scanning is then accessed by a volume renderer 90, which converts the echo signals of a 3D data set into a projected 3D image as viewed from a given reference point as described in U.S. Pat. No. 6,530,885 (Entrekin et al.) The 3D images produced by the volume renderer 90 are coupled to a display processor 92 for further enhancement, graphic overlay, buffering and temporary storage for display on an image display 94.

The operation of the scan converter 88 is illustrated in FIGS. 13 and 14. FIG. 13 depicts pixel or voxel values shown as circles on two acquired scanlines 102 and 104 that were acquired in the vicinity of a final reconstructed image line 110 of display values in the Cartesian coordinates of a grid 100. One widely used image reconstruction technique is to average all pixels within a predetermined distance of a voxel center as shown in FIG. 13, where the value of pixel 112 is averaged with the value of pixel 114 to determine the value of the voxel centered at 106 on line 10 between the two pixels. To reduce motion artifacts due to the slightly different acquisition times of the image data, e.g., beam 102 was acquired in a scan plane with a near field focus and beam 104 was acquired at a different time and in a different scan plane with a far field focus, a more complicated interpolation/reconstruction can be used to introduce different weights for pixels around a reconstructed voxel center according to the distance of the acquired image pixel data from the voxel center. For instance, pixel 114 which is closer to the voxel center on line 110 can be more greatly weighted than the value of pixel 112 which is more distant from the voxel center. This method will reduce the blurring effect in the 3D image since it can provide non-linear pixel value weights.

An even more sophisticated interpolation/reconstruction technique is depicted in FIG. 14. In this illustration, a predefined volumetric region Iₖ is located around the center of each reconstructed voxel location such as voxel center 106. In this example there are six pixels within the volumetric region Iₖ which are weighted to contribute to the display voxel value, three on scanline 102 and three on scanline 104 within the volumetric region. An equation which can be used to calculate each display voxel intensity value is:

$${I}_{new} = \frac{{\sum }_{k = 0}^{n} {W}_{k} {I}_{k}}{{\sum }_{k = 0}^{n} {W}_{k}}$$

where I_{new} is the reconstructed voxel intensity, n refers to the number of pixels that fall within the predefined region, and Wₖ is the relative weight for the kth pixel depending on the distance from the kth pixel to the reconstructed voxel center.

It should be noted that an ultrasound system suitable for use in an implementation of the present invention, and in particular the component structure of the ultrasound system of FIG. 12, may be implemented in hardware, software or a combination thereof. The various embodiments and/or components of an ultrasound system, or components and controllers therein, also may be implemented as part of one or more computers or microprocessors. The computer or processor may include a computing device, an input device, a display unit and an interface, for example, for accessing the internet. The computer or processor may include a microprocessor. The microprocessor may be connected to a communication bus, for example, to access a PACS system or a data network for importing training images and storing the results of clinical exams. The computer or processor may also include a memory. The memory devices such as the image data memory may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or instructions for selecting the proper times or angles of mechanical sweep at which scan planes for a volume image are to be acquired, or the equation to be used for image reconstruction by the scan converter.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine. The set of instructions of an ultrasound system including those controlling the acquisition, processing, and display of ultrasound images and instructions for scan plane acquisition and display volume reconstruction as described above may include various commands that instruct a computer or processor as a processing machine to perform specific operations such as the methods and processes of image data acquisition described above. The set of instructions may be in the form of a software program. The software may be in various forms such as system software or application software and which may be embodied as a tangible and non-transitory computer readable medium. The equation given above for scan converter interpolation and reconstruction is typically calculated by or under the direction of software routines. Further, the software may be in the form of a collection of separate programs or modules within a larger program or a portion of a program module. The software also may include modular programming in the form of object-oriented programming. The processing of input data by the processing machine may be in response to operator commands issued from a control panel, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasonic diagnostic imaging system for three-dimensional (3D) imaging with a mechanical ultrasound probe comprising:
a mechanical ultrasound probe adapted to move an array transducer in a path of movement in an elevation direction;
a beamformer adapted to acquire scan planes of image data at different times and locations along the path of movement;
a scan converter adapted to process the image data to produce voxel values for display in a 3D image; and
a display for displaying the 3D image,
wherein the beamformer is further adapted to acquire a plurality of scan planes of image data with a near field focus and a plurality of scan planes of image data with a far field focus, and
wherein the scan planes with the near field focus are acquired with a separation in the elevation direction which is uniform and which satisfies a selected spatial sampling criterion in the near field, and
wherein the scan planes with the far field focus are acquired with a separation in the elevation direction which is uniform and which satisfies the selected spatial sampling criterion in the far field.

2. The ultrasonic diagnostic imaging system of claim 1, wherein the array transducer further comprises a curved array transducer.

3. The ultrasonic diagnostic imaging system of claim 2, wherein the array transducer further comprises a 1xD array transducer.

4. The ultrasonic diagnostic imaging system of claim 3, wherein the 1xD array transducer has two selectable apertures.

5. The ultrasonic diagnostic imaging system of claim 4, wherein the beamformer adapted to acquire a plurality of scan planes of image data with a near field focus is further adapted to acquire the plurality of scan planes with one of the selectable apertures.

6. The ultrasonic diagnostic imaging system of claim 5, wherein the beamformer adapted to acquire a plurality of scan planes of image data with a far field focus is further adapted to acquire the plurality of scan planes with the other of the selectable apertures.

7. The ultrasonic diagnostic imaging system of claim 1, wherein the plurality of scan plane of image data acquired with a near field focus comprises fewer scan planes than the number of scan planes of image data acquired with a far field focus.

8. The ultrasonic diagnostic imaging system of claim 1, wherein the scan converter is further adapted to process the image data by interpolating received pixel values to produce voxel values for display.

9. The ultrasonic diagnostic imaging system of claim 8, wherein pixel values that are within a predetermined distance of a voxel value center are averaged together.

10. The ultrasonic diagnostic imaging system of claim 8, wherein pixel values that are within a predetermined distance of a voxel value center are averaged together in a weighted average, wherein the weighting is a function of a distance of a pixel from the voxel value center.

11. The ultrasonic diagnostic imaging system of claim 10, wherein the pixel values that are averaged together are located within a predetermined volume centered on a voxel value center.

12. The ultrasonic diagnostic imaging system of claim 1, wherein the mechanical ultrasound probe is further adapted to provide the beamformer with a measure of the location of the array transducer along an elevational path of travel.

13. The ultrasonic diagnostic imaging system of claim 12, wherein the beamformer is responsive to the measure of the location of the array transducer along an elevational path of travel to actuate the array transducer to scan an image plane at predetermined locations in the elevational path of travel.

14. The ultrasonic diagnostic imaging system of claim 13, wherein the elevational path of travel comprises an arcuate path; and
wherein spacings of the locations of acquisition of scan planes of image data with a near field focus along the arcuate path of travel are evenly spaced; and
wherein the spacings of the locations of acquisition of scan planes of image data with a far field focus along the arcuate path of travel are evenly spaced; and
wherein the number of acquisitions of scan planes of image data with a far field focus exceeds the number of acquisitions of scan planes of image data with a near field focus during the time required to acquire a number of scan planes for a 3D volume image.

15. The ultrasonic diagnostic imaging system of claim 2, wherein the disparity between the number of scan planes acquired with a near field focus and the number of scan planes acquired with a far field focus is a function of the radius of curvature of the curved array transducer.

16. A method of 3D ultrasonic imaging with a mechanical ultrasound probe adapted to move an array transducer in a path of movement in an elevation direction, a beamformer adapted to acquire scan planes of image data at different times and locations along the path of movement, a scan converter adapted to process the image data to produce voxel values for display in a 3D image, and a display for displaying the 3D image, the method comprising:
acquiring, by means of the beamformer, a plurality of scan planes of image data with a near field focus, the near field focused scan planes exhibiting a separation in the elevation direction which satisfies a selected spatial sampling criterion in the near field; and
acquiring, by means of the beamformer, a plurality of scan planes of image data with a far field focus, the far field focused scan planes exhibiting a separation in the elevation direction which satisfies the selected spatial sampling criterion in the far field.

17. The method of claim 16, wherein acquiring the near field focused scan planes further comprises acquiring a plurality of scan planes which are uniformly separated in the elevation direction; and
wherein acquiring the far field focused scan planes further comprises acquiring a plurality of scan planes which are uniformly separated in the elevation direction.

18. The method of claim 17, wherein acquiring the near field focused scan planes further comprises acquiring a number of scan planes with the near field focus which is less than the number of scan planes acquired with the far field focus.

19. The method of claim 16, wherein the array transducer further comprises a curved array transducer.
